(19) United States
(12) Patent Application Publication
(10) Pub. No.: US 2025/0152086 A1
(43) Pub. Date: May 15, 2025

(54) DEVICES AND METHODS FOR MONITORING PREGNANCY

(71) Applicant: Nine Medical, Inc., San Francisco, CA (US)

(72) Inventors: Benjamin Kahn CLINE, Palo Alto, CA (US); Ryan Kendall PIERCE, Carl Junction, MO (US)

(21) Appl. No.: 19/022,831

(22) Filed: Jan. 15, 2025

Related U.S. Application Data
(63) Continuation of application No. 17/831,198, filed on Jun. 2, 2022, now abandoned.
Application No. 17/831,198 is a continuation of application No. 16/746,538, filed on Jan. 17, 2020, now abandoned.
Application No. 16/746,538 is a continuation of application No. 15/112,698, filed on Jul. 20, 2016, now abandoned.
(60) Provisional application No. 61/934,427, filed on Jan. 31, 2014.

(51) Int. Cl. A61B 5/00; A61B 5/053; A61B 5/11; A61B 5/391

(52) U.S. Cl. CPC A61B 5/4356; A61B 5/0011; A61B 5/0022; A61B 5/053; A61B 5/11; A61B 5/391; A61B 5/486; A61B 5/6833; A61B 5/721; A61B 5/7275; A61B 5/746; A61B 2562/0219; A61B 2562/14; A61B 2562/242

(57) ABSTRACT

Devices, systems and methods are described herein that enable and make assessments related to pregnancy, including assessments related to a labor-related status such as the onset of true labor. In some embodiments, a pregnancy monitoring system comprises a portable, noninvasive data acquisition device adhered to a patient's skin, comprising at least two electrodes to measure uterine electrical activity, signal processing means, data storage means, and a battery. Systems for notification of the labor-related status are described.

### CROSS REFERENCE TO RELATED APPLICATIONS

This patent application is a continuation of U.S. patent application Ser. No. 17/831,198, titled “DEVICES AND METHODS FOR MONITORING PREGNANCY.” filed Jun. 2, 2022, now U.S. Patent Application Publication No. 2022/0287624, which is a continuation of U.S. patent application Ser. No. 16/746,538, titled “DEVICES AND METHODS FOR MONITORING PREGNANCY.” filed Jan. 17, 2020, now U.S. Patent Application Publication No. 2020/0146614, which is a continuation of U.S. patent application Ser. No. 15/112,698, titled “DEVICES AND METHODS FOR MONITORING PREGNANCY,” filed Jul. 20, 2016, now U.S. Patent Application Publication No. 2016/0331299, which is a U.S. National Phase Application Under 35 U.S.C. § 371 of International Patent Application No. PCT/US2015/014015, titled “DEVICES AND METHODS FOR MONITORING PREGNANCY,” filed Jan. 31, 2015, which claims priority to U.S. Provisional Patent Application No. 61/934,427, titled “DEVICES AND METHODS FOR MONITORING PREGNANCY,” filed Jan. 31, 2014, each of which is herein incorporated by reference in its entirety.

### BACKGROUND

Assessment of a pregnant woman to determine whether delivery will occur within a specific interval, for example within one week, is important to the management of pregnancy, especially in cases of threatened preterm labor. Currently, there is a lack of an accurate assessment means that yields both a high positive predictive value and a high negative predictive value. Additionally, there is a lack of an accurate remote monitoring technology for predicting whether delivery is likely to happen within a specific interval.

### SUMMARY

In one aspect of the present invention, a method of notifying one or more persons of a labor-related status of a pregnant person may involve: populating a register of one or more persons designated for notification of the labor-related status; assessing a likelihood of the labor-related status; comparing the assessed likelihood to a threshold of likelihood of the labor-related status required for notification; and when the assessed likelihood exceeds the threshold of likelihood required for notification, notifying the pregnant person of the labor-related status and notifying one or more persons on the register of the labor-related status.

In some embodiments, the labor-related status may involve one or more of the following: absence of true labor, presence of true labor, onset of true labor, predicted amount of time until onset of true labor, predicted amount of time until the delivery of an infant, and delivery of an infant. In some embodiments, notifying one or more persons on the register may involve delivering to one or more persons on the register, one or more of the following: a text message, an electronic mail, a message within a smartphone application, a social media notification, a phone call.

In some embodiments, the notifying of one or more persons on the register may occur automatically. In some embodiments, the notifying of one or more persons on the register may occur automatically, unless the pregnant person provides a contrary instruction. In some embodiments, the notifying of one or more persons on the register occurs at a time interval after notification of the pregnant person that is designated by the pregnant person.

In some embodiments, the threshold of likelihood may be within the range of 50% to 100%. In some embodiments, the threshold of likelihood may be within the range of 80% to 100%. In some embodiments, the notifying of one or more persons on the register may be delayed until a designated time window of a day. In some embodiments, the designated time window may be selected to avoid waking one or more persons on the register. In some embodiments, the pregnant person may populate the register.

In another aspect, a method of notifying one or more persons of a labor-related status of a pregnant woman may involve: storing contact information for one or more persons, other than the pregnant woman, designated for notification of the labor-related status; electrically coupling at least two electrodes to skin of the pregnant woman; receiving a processed uterine activity signal generated from measured uterine electrical activity; determining based on assessment of the processed uterine activity signal a high likelihood of the labor-related status; and based on the determination of a high likelihood of the labor-related status, delivering a notification to one or more of the persons whose contact information was stored.

In some embodiments, the labor-related status may comprise one or more of the following: absence of true labor, presence of true labor, onset of true labor, predicted amount of time until onset of true labor, predicted amount of time until the delivery of an infant, and delivery of an infant. In some embodiments, delivery of the notification may be automated. In some embodiments, the person to whom the notification is delivered may be selected from the following: a spouse, a partner, a friend, a professional colleague, a family member, and a medical professional. In some embodiments, the notification may comprise one or more of the following: a text message, an electronic mail, a message within a smartphone application, a social media notification, and a phone call.

Some embodiments of the method may further involve following the determination of a high likelihood of the labor-related status, transmitting a wireless communication from a data acquisition device or a patient interface device to initiate transmission of the notification. Some embodiments of the method may further involve mechanically coupling an accelerometer to the pregnant female, receiving motion signals from the accelerometer, calculating a daily number of steps taken by the pregnant female from the motion signals, and notifying the pregnant female of the daily number of steps taken.

In another aspect, a method of monitoring the status of a pregnancy may involve: electrically coupling at least two electrodes to skin of a pregnant female; receiving a processed uterine activity signal generated from measured uterine electrical activity; prompting a user to designate a window of time; storing the window of time; and based on an assessment of the processed uterine activity signal and the designated window of time, determining a likelihood of onset of true labor occurring within the window of time. In some embodiments, the window of time may be set to begin by default at the time at which the window of time is designated.

Some embodiments of the method may further involve mechanically coupling an accelerometer to the pregnant female, receiving motion signals from the accelerometer, calculating a daily number of steps taken by the pregnant female from the motion signals, and notifying the pregnant female of the daily number of steps taken.

In another aspect, a method of assessing the likelihood of a labor-related status may involve: receiving a gestational age of a fetus; electrically coupling at least two electrodes to skin of a pregnant female; receiving a processed uterine activity signal generated from measured uterine electrical activity; and assessing the likelihood of the labor-related status based on the processed uterine activity signal and the gestational age of the fetus.

In some embodiments, the labor-related status may comprise one or more of the following: absence of true labor, presence of true labor, onset of true labor, predicted amount of time until onset of true labor, predicted amount of time until the delivery of an infant, and delivery of an infant. In some embodiments, assessing the likelihood of the labor-related status may utilize the gestational age of the fetus at the time of the assessment.

Some embodiments of the method may further involve adhering the at least two electrodes to the pregnant female with a portable adhesive patch, the adhesive patch comprising a patient-contacting adhesive surface and a processor in electrical communication with the electrodes.

In another aspect, a system for packaging and dispensing an adhesively-secured pregnancy monitoring device may involve: a portable pregnancy monitoring device comprising an adhesive interface, at least one electrode, and a processor; a removable backer covering at least part of the adhesive interface; and a dispenser that substantially covers the portable pregnancy monitoring device and the removable backer.

Some embodiments of the method may further involve a preparatory wipe for preparation of a patient's skin for application of the adhesively-secured pregnancy monitoring device. Some embodiments of the method may further involve an abrading surface for abrading a patient's skin prior to application of the adhesively-secured pregnancy monitoring device. Some embodiments of the method may further involve a plurality of adhesive patches, the adhesive patches comprising an adhesive interface and a removable backer. In some embodiment, the dispenser may involve a cardboard enclosure.

In another aspect, a method of monitoring the status of a pregnancy may involve: adhering to skin of a pregnant female a portable pregnancy monitoring device comprising a processor, at least two electrodes and an adhesive patient-contacting surface; receiving a processed uterine activity signal generated from measured uterine electrical activity; assessing the likelihood of the onset of true labor based on the processed uterine activity signal; and after a period of time of usage has elapsed, removing the pregnancy monitoring device from the skin of the pregnant female, replacing at least an adhesive patient-contacting surface, reusing at least the processor, and re-adhering the pregnancy monitoring device to the skin of the pregnant female.

Some embodiments of the method may further involve replacing at least one electrode that is electrically connected to an interconnection, and attaching an interconnection of a new electrode to an interconnection of a reused portion of the pregnancy monitoring device.

In another aspect, a method of maintaining the substantial position of one or more electrodes used to monitor uterine electrical activity in a pregnant female may involve: coupling the one or more electrodes to skin of the pregnant female using a first adhesive patch; coupling a second adhesive patch to one or more electrodes and to the skin of the pregnant female before removing the first adhesive patch; and removing the first adhesive patch. With this method, the second adhesive patch maintains the substantial position of the one or more electrodes after the first adhesive patch is removed.

In another aspect, a method of maintaining the substantial position of the housing of one or more electrodes used to monitor uterine electrical activity in a pregnant female may involve: coupling the housing of one or more electrodes to skin of the pregnant female using a first adhesive patch; coupling a second adhesive patch to the housing of one or more electrodes and to the skin of the pregnant female before removing the first adhesive patch; and removing the first adhesive patch. With this method, the second adhesive patch maintains the substantial position of the housing of one or more electrodes after the first adhesive patch is removed.

In another aspect, a method for promoting the proper placement of an adhesive patch used to monitor or treat a health-related condition of a mammal may involve: identifying a first edge region of a previously adhered patch; positioning a second edge region of a replacement patch in alignment with the first edge region of the previously adhered patch; and adhering the replacement patch in the aligned position. With this method, the second edge region is configured to substantially match the first edge region.

In some embodiments, the health-related condition may be pregnancy. In some embodiments, the outline of the matching regions of the first and second edge regions may vary from generally concave to generally convex along at least part of its length. In some embodiments, the outlines of edge regions not intended for alignment may not substantially match or interlock. In some embodiments, the aligned position of the second patch may result in substantial adjacency of the first and second edge regions. In some embodiments, the aligned position of the second patch may result in generally parallel tangencies of corresponding sites on the first and second edge regions. In some embodiments, replacement patch may be configured to secure at least one electrode previously secured by the previously adhered patch, while substantially maintaining the at least one electrode's position relative to the mammal.

In another aspect, a method of comparing signals detected by a first and a second pair of electrodes electrically coupled to skin of a pregnant female to monitor uterine electrical activity may involve: applying a given electric potential across an anatomical region generally spanned by a first pair of electrodes electrically coupled to the skin of the pregnant female using a first adhesive patch; recording the impedance detected by the first pair of electrodes; replacing the first pair of electrodes and first adhesive patch with a second pair of electrodes electrically coupled to the skin of a pregnant female using a second adhesive patch; applying the given electric potential across the anatomical region; recording the impedance detected by the second pair of electrodes; and calculating a ratio between the impedances detected by the first and second pairs of electrodes.

In another aspect, a method of comparing signals detected by a first and a second pair of electrodes electrically coupled to skin of a pregnant female to monitor uterine electrical activity may involve: applying a given electric potential across an anatomical region generally spanned by a pair of electrodes electrically coupled to the skin of the pregnant female using a first adhesive patch; recording the impedance detected by the pair of electrodes; replacing the first adhesive patch with a second adhesive patch; applying the given electric potential across the anatomical region; recording the impedance detected by the pair of electrodes; and calculating a ratio between the impedances detected by the pair of electrodes before and after replacement of the first adhesive patch.

In another aspect, a method for assessing the likelihood of a labor-related status of a pregnant female may involve: removing an adhesive patch from a backer; electrically coupling at least two electrodes to skin of the pregnant female using the adhesive patch; receiving a processed uterine activity signal generated from measured uterine electrical activity; measuring a first value of one or more parameters associated with a first processed uterine activity signal; measuring a second value of the one or more parameters associated with a second processed uterine activity signal that occurs a duration of time t after the first processed uterine activity signal; calculating a rate of change in the value of the one or more parameters, by dividing the difference between the second and first values of the one or more parameters by the duration of time t; calculating a difference in the value of the one or more parameters, by subtracting the first value from the second value of the one or more parameters; and assessing the likelihood of the labor-related status based at least in part on one or more of the following: the rate of change, a value derived from the rate of change, the difference, and a value derived from the difference. With this method, the one or more parameters may be chosen from the following list: propagation velocity, peak frequency, amplitude, frequency variance, contractions per unit time, burst duration, burst period, kurtosis, skewness, instantaneous phase, sample entropy, approximate entropy, Tsallis entropy, mutual information, Lempel-Ziv complexity, non-linear correlation coefficient, correlation dimension, fractal dimension, multifractal spectral width, first positive Lyapunov exponent, correlation integral, recurrence percentage, Pearson correlation coefficient, coherence, Granger causality, stochastic event synchrony, synchronization likelihood, and omega complexity.

In some embodiments, the labor-related status may involve one or more of the following: absence of true labor, presence of true labor, onset of true labor, predicted amount of time until onset of true labor, predicted amount of time until the delivery of an infant, and delivery of an infant.

In some embodiments, one or more of the following may be variables utilized in assessing the likelihood of the labor-related status: previous preterm birth by the pregnant female, age of the pregnant female, gestational age of the fetus, whether the pregnant female was prematurely born, gestational age of the pregnant female at the time of her birth, height or weight of the pregnant female, dimensions of the pregnant female's cervix, and results of a fetal fibronectin test performed on the pregnant female. In some embodiments, an accelerometer detecting movement of the pregnant female is used to identify movement artifacts in data collected using the at least two electrodes.

In another aspect, a method for monitoring the status of a pregnancy may involve: electrically coupling at least two electrodes to skin of a pregnant female; receiving a processed uterine activity signal generated from measured uterine electrical activity; identifying contractions from the processed uterine activity signal; and transitioning from a first mode to a second mode after a specified number of contractions have been identified. With this method, transitioning may involve enabling delivery of a notification in response to a subsequent determination of a high likelihood of onset of true labor, wherein delivery of the notification was not enabled in the first mode.

In some embodiments, the specified number of contractions may be within the range of 1 to 15.

Some embodiments of the method may further involve assessing the presence of motion artifacts in the processed uterine activity signal and excluding at least one contraction containing a motion artifact from counting towards the specified number of contractions. Some embodiments of the method may further involve identifying motion artifacts based upon motion signals acquired from an accelerator mechanically coupled to the skin of the pregnant female. Some embodiments of the method may further involve calculating a daily number of steps taken by the pregnant female from the motion signals, and notifying the pregnant female of the daily number of steps taken.

Some embodiments of the method may further involve: following a transition from the first mode to the second mode, determining the likelihood of a labor-related status based at least in part upon one or more of the following: a change in a parameter from a baseline value, a rate of change of the parameter with respect to time, a higher order derivative of the parameter with respect to time, a standard deviation of the parameter. With this method, the parameter may involve one of the following computed from a segment of the processed uterine activity signal: conduction velocity, peak frequency, mean signal frequency, median signal frequency, burst duration, burst period, kurtosis, skewness, instantaneous phase, sample entropy, approximate entropy, Tsallis entropy, mutual information, Lempel-Ziv complexity, non-linear correlation coefficient, correlation dimension, fractal dimension, multifractal spectral width, first positive Lyapunov exponent, correlation integral, recurrence percentage, Pearson correlation coefficient, coherence, Granger causality, stochastic event synchrony, synchronization likelihood, and omega complexity.

In some embodiments, the labor-related status may involve one or more of the following: absence of true labor, presence of true labor, onset of true labor, predicted amount of time until onset of true labor, predicted amount of time until the delivery of an infant, and delivery of an infant. In some embodiments, the segment of the processed uterine activity signal may involve the entirety of the processed uterine activity signal. In some embodiments, the segment of the processed uterine activity signal may involve only portions of processed uterine activity signal identified as contractions. In some embodiments, the processed uterine activity signal containing a motion artifact may be excluded from the segment of the processed uterine activity signal.

### BRIEF DESCRIPTION OF THE DRAWINGS

A number of devices and methods claimed, including some described previously, are illustrated by the following figures.

FIG. 1 illustrates a patch-based data acquisition device worn on the abdomen of a pregnant woman.

FIG. 2 illustrates a schematic drawing showing a data acquisition device.

FIGS. 3, 4, and 5 illustrate pairs of data acquisition devices (including a patch to be replaced, and a replacement patch) that feature geometrically interlocking and/or referencing edges.

FIGS. 6 and 7 illustrate tools that may be used to aid in positioning a data acquisition device onto a wearer.

### DETAILED DESCRIPTION

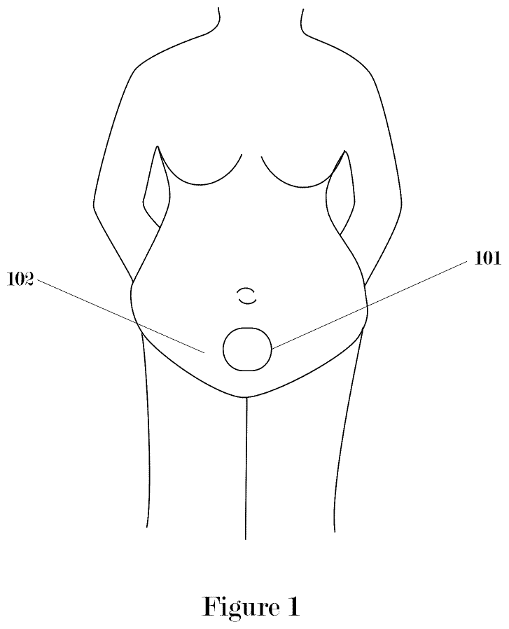

FIG. 1 illustrates an example of a patch-based data acquisition device 101 worn on the abdomen 102 of a pregnant woman. The data acquisition device 101 may be adhered to the abdomen with an adhesive.

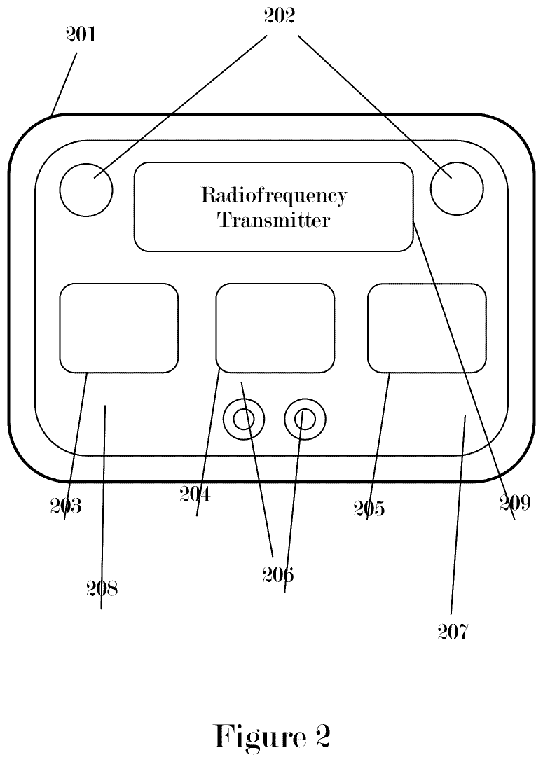

FIG. 2 illustrates a schematic drawing of an example of a data acquisition device for pregnancy monitoring 201, comprised of one or more of the following components: one or more electrodes 202, one or more analog circuits 203, one or more digital circuits 204, one or more batteries 205, one or more LED indicators 206, a flexible housing 207, a flexible circuit board 208, one or more radiofrequency communications modules 209, such as a radiofrequency transceiver. Shown components may be arranged in a variety of configurations not necessarily illustrated in the schematic drawing. An adhesive may aid in securing the data acquisition device to a wearer.

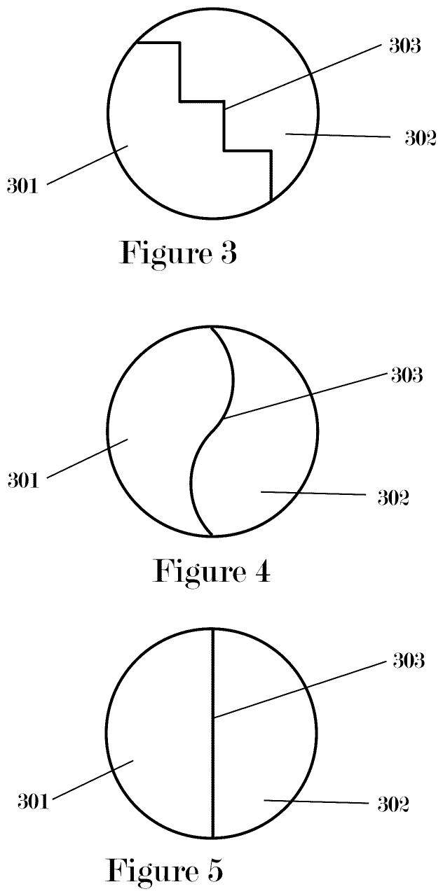

FIGS. 3, 4, and 5 illustrate examples of pairs of data acquisition devices (including a patch to be replaced 301, and a replacement patch 302) that feature geometrically interlocking and/or referencing edges 303. Edges 303 help designate the position of replacement patch 302 relative to a patch to be replaced 301, and may promote patch positions (and/or electrode positions) intended to advantageously detect uterine electrical activity or provide a ground electrode for detection of said activity. Said referencing may also be achieved using graphics, colors, and/or text that indicates a proper location of one patch to another. In some cases, the shape or location of the interlocking and/or referencing edges 303 also provide direction reference that aids in placement, or contributes to a resemblance to a logo or icon.

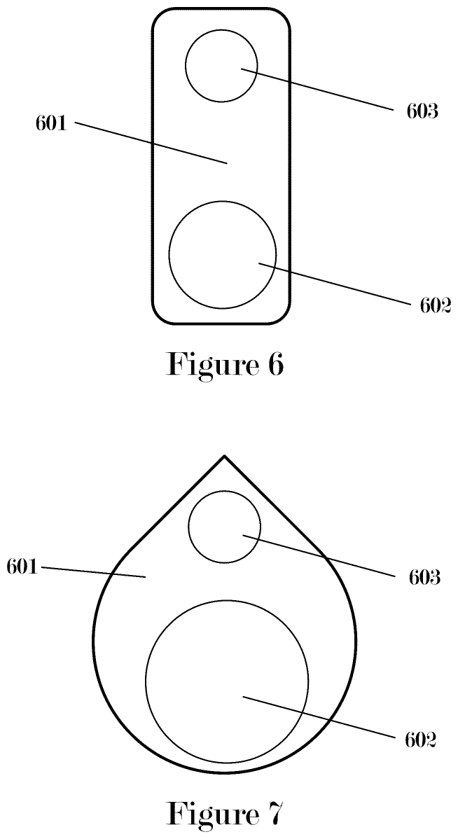

FIGS. 6 and 7 illustrate tools 601 that may be used to aid in positioning a data acquisition device 602 onto a wearer. In some embodiments, a navel reference hole 603 is provided and intended for juxtaposition over a navel; proper said juxtaposition promotes positioning of a patch-based data acquisition device at a desired location. Said hole may be replaced with a notch, indentation, arrow, partial hole, or other feature that provides adequate reference to one or more anatomical features of said wearer.

The devices, methods, and systems shown and discussed above and in FIGS. 1-7 may be used in various combinations. Various additional devices, methods, and systems that incorporate and utilize those discussed above are described below.

The devices and methods described herein utilize a pregnancy monitoring system to make assessments related to pregnancy, such as the likelihood of labor and/or delivery within a specified length of time or before a specified gestational age. In some embodiments, assessment is made of a labor-related status, including but not limited to one or more of the following: absence of true labor, presence of true labor, onset of true labor, predicted amount of time until onset of true labor, predicted amount of time until the delivery of an infant, and delivery of an infant. In some cases, a likelihood of a labor-related status is determined and/or assessed, and provided to a pregnant female, a medical professional, a spouse or partner, a professional colleague, a friend, a family member, a hospital, and/or another individual. In some cases, said likelihood may be expressed or used as one or more of the following: a percentage likelihood, a range of percentage likelihoods, a binary determination, and a descriptive term, such as “high” or “low.” In many embodiments, the pregnancy monitoring system comprises a portable, noninvasive data acquisition device adhered to the patient's abdomen, comprising at least two electrodes to measure uterine electrical activity, signal processing means, data storage means, and a battery. Such a device may be worn continuously by the patient for an extended period of time.

In some embodiments, uterine electrical activity measured utilizing two or more electrodes electrically to the skin of a pregnant female is converted from an analog signal to a digital signal by an analog to digital converter. In some embodiments, processed uterine activity signal comprises the converted signal. In some embodiments, the converted signal is filtered based on frequency; for example, a bandpass with corner frequencies of 0.3 Hz and 3 Hz may be applied. In some embodiments, processed uterine activity signal comprises the filtered signal. In some embodiments, processed uterine activity signal comprises a signal derived from the converted signal.

In some cases, the pregnancy monitoring system comprises a data acquisition device comprising at least two electrodes for measuring uterine electrical activity, means for processing data from said electrodes, means for assessing or helping to assess whether labor and/or delivery is likely to occur within a specified length of time or before a specified gestational age, and means to indicate to the patient whether labor and/or delivery is likely to occur within a specified length of time or before a specified gestational age. Such a data acquisition device may be a patch with one or more adhesive surfaces to bond to the belly of a pregnant woman. A data acquisition device may also be referred to as a pregnancy monitoring device.

In some cases, the pregnancy monitoring system comprises a data acquisition device and a remote center. The data acquisition device comprises at least two electrodes for measuring uterine electrical activity, means for processing data from said electrodes, and a means to communicate with said remote center, for example using a cellular telephone network (for example, utilizing the Global System for Mobile Communications standard). The remote center comprises at least one processor and means to communicate with said data acquisition device, for example via a cellular telephone network or internet connection. The remote center may comprise a distributed processing center or backend server. Data transmission between the data acquisition device and the remote center occurs between once a second to once every 10 days, more preferably between once a minute and once a day.

The data acquisition device may perform computation on acquired signal data and generate a derived data set. Such a data acquisition device transmits one or more of the following types of data to the remote center: some or all of the acquired signal data, and/or some or all of the derived data. Assessment of data (for example to determine the likelihood of delivery) and determination of whether notification (for example of the pregnant woman and/or her physician) is necessary may be performed by the data acquisition device, and/or the remote center and/or an individual such as the pregnant woman's physician who is provided access to acquired and/or derived data (such as access via the remote center). Said assessment and determination to generate a notification may be conducted in an automated of semi-automated process, and/or may be conducted by a person. If the determination is made that notification is necessary, said determination may be communicated by the data acquisition device, and/or the remote center to other component(s) of the pregnancy monitoring system and/or notifications may delivered to individuals and entities selected as notified parties. Details pertaining to notifications, including notified parties and methods of notification, are described in the notifications section.

In some cases, the pregnancy monitoring system comprises a data acquisition device and a patient interface device. The data acquisition device comprises at least two electrodes for measuring uterine electrical activity, means for processing data from said electrodes, and a means to communicate with said patient interface device, for example using radiofrequency communication (such as using the Bluetooth Low Energy standard). The patient interface device has at least one processor, means to communicate with said data acquisition device, for example using radiofrequency communications, and a means to deliver a notification to the pregnant woman, for example that labor and/or delivery is likely to occur within a specified length of time or before a specified gestational age. In some cases, the patient interface device has a rechargeable battery. Said patient interface device may comprise the pregnant woman's cellular telephone, or another person's phone (including the phone of a person often in close proximity with the patient), or dedicated electronic wristband device. Data transmission between the data acquisition device and the patient interface device occurs between once a second to once every 10 days, more preferably between once a minute and once a day.

Said patient interface device may utilize an application. Said application may be downloaded for the purpose of use with, or as part of, the pregnancy monitoring system. Said application may be configured to communicate with a data acquisition device. Said application may be synced to a particular data acquisition device (or set of such devices); syncing may be achieved by entering information, such as a code, provided with the data acquisition device.

The data acquisition device may perform computation on acquired signal data and generate a derived data set. Said data acquisition device transmits one or more of the following types of data to the patient interface device: some or all of the acquired signal data, and/or some or all of the derived data. Assessment of data (for example to determine the likelihood of delivery) and determination of whether notification (for example of the pregnant woman and/or her physician) is necessary may be performed by the data acquisition device, and/or the patient interface device. Said assessment and determination to generate a notification may be conducted in an automated of semi-automated process, and/or may be conducted by a person. If the determination is made that notification is necessary, said determination may be communicated by the data acquisition device, and/or patient interface device to other component(s) of the pregnancy monitoring system and/or notifications may delivered to individuals and entities selected as notified parties. Details pertaining to notifications, including notified parties and methods of notification, are described in the notifications section.

In some cases, the pregnancy monitoring system comprises a data acquisition device, a patient interface device, and a remote center. The data acquisition device comprises at least two electrodes for measuring uterine electrical activity, means for processing data from said electrodes, and a means to communicate with said patient interface device, for example using radiofrequency communication (such as using the Bluetooth Low Energy standard). The patient interface device has at least one processor, means to communicate with said data acquisition device, for example using radiofrequency communications, means to communicate with said remote center, for example using a cellular telephone network (for example, utilizing the Global System for Mobile Communications standard), and a means to deliver a notification to the pregnant woman, for example that labor and/or delivery is likely to occur within a specified length of time or before a specified gestational age. In some cases, the patient interface device has a rechargeable battery. Said patient interface device may comprise the pregnant woman's cellular telephone, or another person's phone (including the phone of a person often in close proximity with the patient), or dedicated electronic wristband device. The remote center comprises at least one processor and means to communicate with said patient interface device, for example via a cellular telephone network or internet connection. The remote center may comprise a distributed processing center or backend server. Data transmission between the patient interface device and the remote center, and data transmission between the data acquisition device and the patient interface device occurs between once a second to once every 10 days, more preferably between once a minute and once a day.

Said patient interface device may utilize an application. Said application may be downloaded for the purpose of use with, or as part of, the pregnancy monitoring system. Said application may be configured to communicate with a data acquisition device and/or a remote center. Said application may be synced to a particular data acquisition device (or set of such devices), and/or remote center; syncing may be achieved by entering information, such as a code, provided with the data acquisition device.

The data acquisition device may perform computation on acquired signal data and generate a derived data set. Said data acquisition device transmits one or more of the following types of data to the patient interface device: some or all of the acquired signal data, and/or some or all of the derived data. Said patient interface device may perform computation on data received from said data acquisition device and generate a derived data set. Said patient interface device transmits one or more of the following types of data to the remote center: acquired signal data received from the data acquisition device, and/or derived data received from the data acquisition device, and/or data derived by said patient interface device. Assessment of data (for example to determine the likelihood of delivery) and determination of whether notification (for example of the pregnant woman and/or her physician) is necessary may be performed by the data acquisition device, and/or the patient interface device, and/or the remote center and/or an individual such as the pregnant woman's physician who is provided access to acquired and/or derived data (such as access via the remote center). Said assessment and determination to generate a notification may be conducted in an automated of semi-automated process, and/or may be conducted by a person. If the determination is made that notification is necessary, said determination may be communicated by the data acquisition device, patient interface device, and/or the remote center to other component(s) of the pregnancy monitoring system and/or notifications may delivered to individuals and entities selected as notified parties. Details pertaining to notifications, including notified parties and methods of notification, are described in the notifications section.

In some cases during the course of the patient's usage of the pregnancy monitoring system, the patient may use the same patient interface device and/or remote center, while replacing her data acquisition device one or more times. In some cases, when the pregnancy monitoring system is initially set up for use, the data acquisition device may be paired with a patient interface device and/or remote center. In some cases, when the data acquisition device is replaced, the patient interface device and/or remote center can re-pair with the new data acquisition device. In some cases, after re-pairing has been established, the patient interface device may transmit information to the new data acquisition device. Such information may include calibration data, raw data acquired by previous data acquisition device(s), processed data based on data from previous data acquisition device(s), and patient information.

In some embodiments, the data acquisition device contains at least one feature to assist in proper alignment to the patient's body. For example, said alignment assistance feature can include a mark or hole that is aligned with the patient's belly button; arrow(s) to indicate which direction on said data acquisition device faces upwards towards the patient's head; printed text to indicate which direction on said data acquisition device faces upwards towards the patient's head; an illustration indicating which direction on said data acquisition device faces upwards towards the patient's head.

In some cases, the pregnancy monitoring system includes a template that is used to align a patch-like device to the patient. Said template may contain indications of anatomical landmarks, for example the patient's navel. Said template may be used to align a replacement patch-like device relative to a previously used patch-like device.

In some cases, the data acquisition device of the pregnancy monitoring system comprises a patch-like device and a backer. Once an adequately correct location is found on the patient for said data acquisition device, such a location can be maintained while part or all of the backer is removed. For example, such a backer may contain multiple removable regions; after one removable region is removed, a sticky region of the patch-like device is exposed. The sticky region can hold the patch-like device in place while other regions of the backer are removed and the patch-like device is fully adhered.

In some cases, the pregnancy monitoring system comprises a data acquisition device that is held at a consistent location on the patient's abdomen using one or more straps. Such straps may be clastic straps or non-clastic straps.

In some cases, the pregnancy monitoring system comprises at least two electrodes that are incorporated into the patient's clothing and thus held at a consistent location on the patient's abdomen.

In some cases, the data acquisition device of the pregnancy monitoring system comprises at least one patch-like-device, for example a flexible elastomeric patch containing electrodes that attaches to the patient's skin using an adhesive. In some cases, the patch-like devices are replaced during the course of use of the pregnancy monitoring system, and contain features, such as partial or full edges that geometrically interlock with a partial or full edge of a replacement patch-like device, that indicate to the patient or other person where to place the replacement patch-like device. In some cases, said interlocking results in positioning of one or more electrodes of a replacement patch-like device that mirrors the positioning of one or more electrodes of the replaced patch-like device relative to a median plan of the pregnant woman. In some cases, placement of subsequent replacement patch-like devices does not fully overlap and/or does not overlap at all the position of the replaced patch-like device(s).

In some cases, the pregnancy monitoring system comprises a data acquisition device comprising one or more patch-like-device that attaches to the patient's skin using an adhesive. In some cases, the patch-like device(s) are replaced during the course of use of the pregnancy monitoring system.

During the replacement process for a period of time both the old and new patch may be placed on the patient's body. The data acquired during the time that both patches are applied may be used to calibrate the new patch. For example, a magnitude of a measurement of a signal corresponding to a physiological parameter measured by the new patch, compared with the magnitude measured by the old patch, may be used to achieve a consistent understanding or measurement of the physiological parameter that spans patches used sequentially, even if the old and new patches differ in sensitivity to the signal.

Replaced and replacement patch-like device(s) may each contain the same number of electrodes. Such electrodes may be geometrically configured in a substantially equivalent manner in each of said patch-like devices used during the course of use of said pregnancy monitoring system. For example, the interelectrode spacing may be substantially equivalent between successive patch-like devices.

In some cases, the pregnancy monitoring system contains a data acquisition device with two or more electrodes. Such a device may be placed on the surface of a pregnant woman's abdomen, for example to acquire uterine electrical activity data. Such a device may be placed on or centered mediolaterally with respect to the uterine median plane and/or the median plane of the patient's abdomen (for example, using the patient's navel as an anatomical landmark). Such device may be placed between and/or centered in the inferior-superior axis between the uterine fundus and the pubic symphysis; or the navel and the pubic symphysis. In some cases, the pregnancy monitoring system contains at least two electrodes positioned on the median vertical axis of the patient's abdomen.

In some cases, the pregnancy monitoring system comprises a data acquisition device comprising at least one patch-like-device attaches to the patient's skin using an adhesive. In some cases, said adhesive is partially or fully covered by a removable backer until said device is ready for use. In some cases, said adhesive is reusable. For example, if the patient's physician is not satisfied by the initial placement of said device, he or she may remove said device and re-adhere it to the patient in the proper location. In some cases, said adhesive is breathable. In some cases, said device contains at least one geometric feature that aids in removing said device. Said geometric feature may comprise a pull tab. In some cases, said adhesive is a pressure sensitive adhesive, for example, a silicone, a polyacrylate, or a polyisobutylene, pressure sensitive adhesive. In some cases, said adhesive is conductive in at least some regions.

In some embodiments, the pregnancy monitoring system comprises a data acquisition device configured to minimize mechanical stress and/or strain in the abdominal region arising from the data acquisition device's placement, as in some cases might arise from expansion of the abdominal surface during the course of pregnancy. Such a stress-minimizing configuration may be achieved by with one or more of the following: i) using one or more materials selected for low stiffness, durometer, or Young's modulus, and ii) a geometry selected for low stiffness, such as a geometry with a thin cross section. Such a stress-minimizing configuration can include a patch that has cuts and/or discontinuities and/or a geometry that allow the sticky sites to remain in place, while other regions can migrate. Such a stress-minimizing configuration can include slits to accommodate expansion and/or a pre-compressed patch compressed in the plane of the belly.

In some embodiments, the pregnancy monitoring system contains a feature such as a strain gauge that measures the change in distance between at least two electrodes. After the change in distance has been determined to have exceeded a preset threshold, the pregnancy monitoring system may deliver a notification that the data acquisition device is no longer functioning optimally and/or should be replaced (for example, said indication may be delivered via text message to the patient's mobile phone).

In some embodiments, the pregnancy monitoring system is configured to track the position of at least two electrodes. The position of the electrodes is then utilized in an algorithm extracting features from uterine electric activity.

In some embodiments, the data acquisition device is intended to be used for a sufficiently short time that changes in the patient's belly surface will not have consequential impact on the distance between electrodes and/or on analysis based on the assumption of a fixed distance between electrodes or on the integrity of adherence of the device to the skin. The patient may be instructed to replace the data acquisition device at a regular interval, for example between every week and every month.

In some cases, the pregnancy monitoring system may include a device that exists in a sleep mode until properly placed on the patient's belly. Said sleep mode conserves battery power. In some cases, temperature, removal of an adhesive backer, removal from a sealed pouch, impedance between electrodes, insertion of a battery, and/or presence of a cardiac signal, or another predetermined input and/or trigger is used to determine whether said device has been placed on the patient's belly and/or will soon be put into use. In some cases, said device wakes from said sleep mode to check for said predetermined input and/or trigger; in some cases, said predetermined input and/or trigger triggers waking from sleep mode. After said predetermined sensor input is obtained, said device in some cases automatically enters the mode for monitoring the patient's pregnancy, and/or in some cases enters a mode of preparation and/or readiness for calibration and/or syncing with other components of the pregnancy monitoring system, and/or in some cases enters a different state in a state machine.

In some cases, the pregnancy monitoring system contains a user-input component such as a push button, for the patient, patient's physician, or other person to turn the system on and trigger the beginning of monitoring. The pregnancy monitoring system may provide feedback of successfully turning on and/or beginning monitoring, such as a flashing light emitting diode.

In some cases, the data acquisition device of the pregnancy monitoring system will wake from its sleep state and begin acquiring signal data after it has received an activation signal from a patient interface device. For example, a patient may activate said data acquisition device wirelessly from a cellular telephone.

In some cases, two or more data acquisition devices are used simultaneously, and/or in sequence. For example, one or more first data acquisition devices may be replaced with one or more subsequent data acquisition devices.

In some cases, the pregnancy monitoring system comprises a data acquisition device comprising at least one electrode and a surface configured to contact the patient's skin. Said surface may be non-planar in an unstressed condition. For example, in an unstressed condition, said surface may be arced or may form a spherical section, in order to better conform to the shape of the patient's abdomen. Said device may be substantially oblong to avoid challenges of sticking to a surface that is non-planar. Said device may be shaped like a logo. Such a device may be shaped like a baby.

In some cases, the data acquisition device may comprise a patch with at least one electrode to be adhered to a pregnant woman's abdomen. Said patch may be manufactured in a variety of hues to correspond with a range of flesh pigmentations.

In some cases, the data acquisition device may include at least one design feature on the outer surface, such as text and/or graphics, which is difficult to see when the device is worn under the woman's clothing. For example, said design feature may be a similar color and brightness to the background color of said device, may be in a skin tone color, may be partly or fully transparent, may be embossed in said device, or may be debossed in said device.

In some cases, the colors, shapes, patterns, and/or graphics featured on replacement patches may differ from those featured on replaced patches. These differences may achieve one or more purposes, including but not limited to: aesthetic variety, and/or communication of pregnancy-related information (such as gestational age, weeks remaining until full-term and/or anticipated birth, fetal development), and/or contact information for a medical professional and/or the manufacturer of the patch and/or customer service personnel. Medical professionals and/or wearers of said patch may also be able to choose among multiple options for patch color, shape, patterns, and/or graphics.

In some cases, analysis of acquired signal data, determination of whether a notification needs to be delivered, and delivery of the notification occurs in essentially real time. For example, in some cases, analysis of acquired electrical uterine activity data, determination of whether delivery is likely to occur within a specified length of time or before a specified gestational age, and if delivery is likely to occur, notification of the patients, occurs within 10 minutes of data acquisition (and in some cases, within 1 second; in some cases, within 1 millisecond).

In some cases, the pregnancy monitoring system predicts whether delivery is likely to occur within a specified length of time or before a specified gestational age. In some cases, uterine electrical activity data used for said prediction is filtered to remove artifacts including one or more of: movement artifacts, which may be identified using a movement sensor such as an accelerometer; fetal heart beat; maternal heart beat. In some cases, said filtering utilizes adaptive filtering, Kalman filtering, regression filtering, and/or blind source separation. Preprocessing including wavelet transform may be applied to signals. Delay differential equations may be applied to signals for feature extraction. In some cases, signals are derived from linear combinations of signals from surface uterine activity electrodes. In some cases, said prediction is based on all time periods of uterine electrical activity data. In some cases, said prediction is based on a subset of uterine electrical activity data. In some cases, said prediction is based on analysis of uterine electrical activity data for time periods identified as contractions. In some cases, identification of contractions is automatically computed. In some cases said identification is based on estimation of intrauterine pressure using uterine electrical activity data. In some cases, said identification is based on a tocodynamometer, which may contain a pressure transducer. Features used for said prediction may include spatiotemporal, linear, nonlinear, synchrony, and/or complexity features. More specifically, said features may include conduction velocity, angle of propagation, mean frequency, median frequency, peak frequency, fast wave high frequency, fast wave slow frequency, burst duration, burst period, distribution of amplitude values (for example, kurtosis and/or skewness), instantaneous phase, sample entropy, approximate entropy, Tsallis entropy, mutual information, Lempel-Ziv complexity, non-linear correlation coefficient h², correlation dimension d2, fractal dimension, multifractal spectral width, first positive Lyapunov exponent, correlation integral, recurrence percentage, Pearson correlation coefficient, coherence, Granger causality, stochastic event synchrony, synchronization likelihood, omega complexity, the S-estimator, the S—H—N-indices, mean phase coherence, and/or Hjorth Parameters. Features may be derived from features listed above, including change from baseline, rate of change, standard deviation, higher order derivatives, and ratios between features. One or more of the following classifiers may be applied: linear discriminant classifier, quadratic discriminant classifier, uncorrelated normal density classifier, polynomial classifier, logistic classifier, K nearest neighbor classifier, decision tree classifier, Parzen classifier, support vector classifier, neural network classifier, gray relational analysis classifier. For example, a support vector classifier may be used for binary classification of the state of likely to deliver within a preset time or the state of not likely to deliver within said preset time.

The pregnancy monitoring system may integrate clinical data, such as gestational age, number of fetuses, body mass index of the mother, parity of the mother, whether the mother has had previous preterm deliveries, cervical remodeling (including measures of cervical length, softness, ripening, and/or effacement), maternal respiration rate, maternal heart rate, and/or fetal heart rate into the assessment of the likelihood of labor and/or delivery within a specified length of time or before a specified gestational age. For example, a patient, patient's physician, or other person may enter said clinical data into an electronic interface, such as a smartphone application. Additionally, the pregnancy monitoring system may contain means for assessing parameters such as maternal respiration rate (for example, using an accelerometer), maternal heart rate (for example, using cutaneous electrodes), fetal heart rate (for example, using cutaneous electrodes), maternal stress (for example, by measuring galvanic skin response, maternal respiration rate, and/or maternal heart rate), maternal temperature (for example, using a cutaneous temperature sensor), maternal posture (for example, using an accelerometer), maternal activity (for example, using an accelerometer), maternal steps taken (for example, using an accelerometer) and/or maternal fluid status (for example, by measuring skin impedance). Parameters such as these may displayed to the pregnant woman and/or to another user, and/or status regarding parameters such as these may be reported via a notification. For example, a daily step count of the pregnant woman may be derived from data collected with an accelerometer that is part of the data acquisition device, and the daily step count may be displayed to the pregnant woman on the patient interface device.

The pregnancy monitoring system may include time-keeping means. The pregnancy monitoring system may periodically update the gestational age based on the gestational age initially entered, and the time elapsed since that entry. Based on said updated gestational age, the pregnancy monitoring system may readjust parameters related to assessment of the likelihood of labor and/or delivery within a specified length of time or before a specified gestational age.

The pregnancy monitoring system may collect and use one or more of a variety of data parameters. For example, the pregnancy monitoring system may collect and use fetal movement, which may be measured with an accelerometer. Fetal movements may be distinguished from movements external to the uterus based on the direction of acceleration, as fetal movements are expected to push outwards relative to the mother's belly's surface. Two or more motion detection sensors such as accelerometers may be applied in different locations to distinguish fetal movement from maternal movement. In another example, the pregnancy monitoring system may collect and use kicking movements, which may be measured with an accelerometer. Kicking movements may be distinguished from movements external to the uterus based on the direction of acceleration, as kicking movements are expected to push outwards relative to the mother's belly's surface. In another example, the pregnancy monitoring system may collect and use uterine contractions, which may be measured by assessing belly surface electrical activity (electrohysterography) and/or by using a pressure sensor to assess movement of the belly surface (external tocodynamometry). In another example, the pregnancy monitoring system may collect and use fetal heart rate, which may be assessed using noninvasive fetal electrocardiogramaxample abdominal fetal electrocardiogramd/or using ultrasound, for example pulsed Doppler ultrasound. In additional examples, the pregnancy monitoring system may collect and use one or more of the following: fetal sleep or wakefulness status; maternal sleep or wakefulness status; fetal orientation; maternal respiration rate; correlation of fetal heart rate and/or fetal movement, including fetal heart rate and/or movement triggered by acoustic and/or vibroacoustic stimulation; correlation of contractions and fetal heart rate; maternal skin temperature; data collected using a strain gage, for example a strain gage incorporated into the pregnancy monitoring system. The system may deliver said data to the patient and/or the patient's physician, as well as derived metrics including frequency and trends in occurrence.

At least one algorithm used by the patient monitoring system may adapt based on input data for a specific patient. For example, after placement on the patient's belly, the pregnancy monitoring phase may first undergo a training phase. The pregnancy monitoring system may adjust pregnancy assessment parameters based on this training phase for a subsequent active monitoring phase. In some cases, training and active monitoring phases may partly or fully overlap.

In some embodiments, the pregnancy monitoring system makes at least one transition between states as part of operation. After application to the abdomen of a pregnant woman, the pregnancy monitoring system may initially gather data regarding uterine activity as part of a baseline assessment state. After gathering a sufficient amount of baseline data, the system may transition to an active monitoring state. For example, the pregnancy monitoring system may acquire data corresponding to a certain number of contractions prior to transitioning to the active monitoring state. Prior to completing a baseline assessment, the pregnancy monitoring system may not have sufficient data to accurately estimate the likelihood of a labor-related status such as the onset of true labor. Preventing the delivery of a notification in the baseline assessment state may help prevent an inaccurate notification regarding a labor-related status from being delivered. Transitioning to the active monitoring state may involve enabling notifications that were not enabled in the baseline assessment state. In some embodiments, when the pregnancy monitoring system is operating in the active monitoring state and detects a contraction, the pregnancy monitoring system may determine the likelihood of a labor-related status based on comparison of the contraction to one or more contraction detected in the baseline assessment state; if the likelihood is determined to be above a threshold, a notification may be delivered. The term baseline assessment mode may be used to describe the baseline assessment state; the term active monitoring mode may be used to describe the active monitoring state.

The pregnancy monitoring system may include a state machine that transitions between states. In some embodiments, the pregnancy monitoring system comprises at least two electrodes coupled to a pregnant female, a processor configured to analyze uterine activity, a notification delivery system in a first notification mode, and a notification manager configured to compare received uterine activity data to a threshold, and if the uterine activity data exceeds the threshold, transition the notification delivery system to a second notification mode, wherein at least one additional type of notification is enabled. In some embodiments, the uterine activity data compared to the threshold, comprises contractions; once a certain number of contractions have been received, the transition occurs.

In some cases, the pregnancy monitoring system stores in memory all acquired signal data. In some cases, the pregnancy monitoring system stores in memory a selection of acquired signal data, for example selections of acquired signal data that correspond to contractions. In some cases, the pregnancy monitoring system stores processed analysis of acquired signal data and/or derived data. Locations for said storage include a data acquisition device, a patient interface device, and a remote center. Said data may be stored at multiple locations within the pregnancy monitoring system simultaneously.

In some cases, the pregnancy monitoring system provides notification to a pregnant woman, a medical professional, a medical organization, a company, or one or more individuals, for example friends or family members selected or approved by the woman whose pregnancy is being monitored. Notification should be understood to include indication of a status (including an unchanged status, and/or a status that does not require action, which could include indication that preterm labor or birth is not imminent).

The pregnancy monitoring system may utilize one of more of the following types of notification: notification of low battery (of any component of the pregnancy monitoring system, including the data acquisition device, and patient interface devices such as the patient's cellular telephone); notification of full data storage or limited room to store new data; notification of the need to replace a component of the pregnancy monitoring system (for example, due to a low battery in a data acquisition device, due to a prespecified time having elapsed since application of said data acquisition device, due to stage of pregnancy, due to strain within the data acquisition device, due to quality of acquired signal(s), and/or due to the state of the adhesive of the data acquisition device). The pregnancy monitoring system may utilize notification of communications channel status between components of the pregnancy monitoring system; said communications channel may, for example, be between a data acquisition device and a patient interface device, or between a patient interface device and a cellular telephone network. Notifications may communicate whether the communication channel is functioning properly, whether the communication channel has been disrupted, and/or whether the communication channel has been disrupted for more than a prespecified period of time.

The pregnancy monitoring system may contain an affirmative visual status indicator that indicates to the patient, patient's physician, or other person that the system is functioning properly. For example, the data acquisition device may contain a green LED that is continuously on or flashes when the system is functioning properly and is off when the system is not functioning properly. In some cases, components of the pregnancy monitoring system such as the data acquisition device contain an affirmative visual status indication that indicates to the patient, patient's physician, or other person that said component is functioning properly.

In some cases, the pregnancy monitoring system provides an indication regarding whether the data acquisition device has been properly applied. In some cases, after initial placement the data acquisition device ascertains whether a predetermined signal, such as the cardiac cycle or components thereof, can be acquired from one or more electrodes. In some cases, based on determination of proper placement, the data acquisition system displays a visual signal, such as a green blinking LED, or an LED that fades between brighter and dimmer levels. In some cases, based on determination of improper placement, the data acquisition system displays a visual signal, such as a red LED. In some cases, the pregnancy monitoring system provides an indication regarding whether the data acquisition device has been properly applied via a patient interface device, such as the patient's cellular telephone. In some cases, the pregnancy monitoring system provides an indication regarding whether signal acquisition currently is and/or has been adequate.

In some cases, the pregnancy monitoring system provides an indication of whether labor and/or delivery is likely to occur within a specified length of time or before a specified gestational age. In a number of examples, such length of time may be between 1 hour and 120 days, especially 1 day and 30 days. Said length of time may include 1 day, 2 days, 3 days, 4 days, 5 days, 6 days, 7 days, 10 days, 14 days, 21 days, and 30 days. Said gestational age may be between 18 and 44 weeks, especially 24-40 weeks. Said indication may be accompanied by an assessment of the uncertainty regarding the prediction. In some cases, the pregnancy monitoring system indicates the estimated time to delivery. Notifications may represent the likelihood of the onset of delivery and/or whether labor and/or delivery is likely to occur within a specified length of time or before a specified gestational age with: a) a quantitative metric (for example a percentage) and/or b) a qualitative metric (for example, a color coded level selected on a spectrum containing green, yellow, and red).

The pregnancy monitoring system may send some or all of the following messages and information: report of activity detected; comparison of activity detected to baseline and/or normal levels; messages of encouragement; tips for a healthy pregnancy; promotion of products or services.

Notifications may be visual in nature, and may include, for example, one or more of the following: a color-coded LED on the data acquisition device; display of a quantitative delivery likelihood metric on a patient interface device; display of a graph indicating delivery likelihood on a patient interface device; delivery of nomogram on a patient interface device. Different notifications may be represented by visual indicators with different colors, shapes, symbols, frequency of activation, and/or patterns in on-off activation sequence.

Notifications may be audio in nature, and may include, for example, an audio message delivered by a speaker in a data acquisition device adhered to the patient's abdomen, and/or an automated telephone message delivered to the patient's physician's office telephone. Different notifications may be represented by different audio alarm patterns.

Notifications may be tactile in nature, delivered for example by a vibration in a data acquisition device adhered to the patient's abdomen or patient interface device.

A notification may comprise an electronic message, for example delivered via text message to the patient's cellular telephone or the patient's obstetrician's cellular telephone, or via email to the patient's obstetrician.

In some cases, for example if delivery is likely to occur within a specified length of time or before a specified gestational age, a patient and/or another designee may be notified to contact her physician.

In some cases, for example if delivery is likely to occur within a specified length of time or before a specified gestational age, the patient's physician will be notified, for example by email, text message, fax, or phone call. In some cases, if an acknowledgement is not received from the physician within a specified period of time, then an alert may be sent to an alternative physician, a hospital, and/or to the patient directly.

Delivery of notification may vary depending on the gestational age, estimated time to delivery, and/or time interval within which delivery is likely. Said variation may include the route of notification (for example, audio versus video), the timing of the notification (whether there is a delay due to time of day), and/or who is notified. For example, if at 4 a.m. local time the pregnancy monitoring system determines that delivery is likely within a preset threshold of 14 days, and the estimated time to delivery is 13.99 days, then the indication may be delayed until 8 am.

Delivery of notification may vary depending on the local time of day. For example, if a time-critical notification is to be delivered to the patient at 3 am local time, a vibration or audio signal may be used instead of a visual signal.

Delivery of said notification may depend on ambient conditions. For example, the pregnancy monitoring system may contain a photodiode to measure ambient light and a light emitting diode that flashes to indicate that the device is functioning properly. The brightness of the light emitting diode may depend on the ambient light signal; for instance, said light emitting diode indicator may flash less brightly under clothing or in a dark room when the patient may be trying to sleep.

Notification may be based on a persistence warning framework, wherein said notification once triggered is maintained until manually reset, for a preset length of time, and/or until a debouncing algorithm has determined that said notification should be reset.

Notification may be provided in advance of when said required action shall be taken, or at a time action should be taken.

In some cases, the pregnancy monitoring system may transmit information to one or more individuals via social media, such as Facebook or Twitter.

Notifications may be delivered to the patient, the patient's physician, other individuals selected by the patient, other individuals or institutions selected by the patient's physician, the patient's hospital, the company that markets the pregnancy monitoring system, an affiliate of the company that markets the pregnancy monitoring system, and/or a customer service center.

Notification may be delivered to a pre-specified list of contacts.

In some cases, the pregnancy monitoring system may enable the patient, the patient's physician, or other person, to select who is eligible to receive notifications. In some cases, said system may enable selection of which notifications are to be received by each member of the notification list, how the notifications are to be delivered to each member, any desired delays in notification timing and/or desired time of day for notification of each member. For example, a patient may choose to have her partner, parents, primary care physician, and obstetrician notified when delivery is likely to occur within a specified length of time.

The pregnancy monitoring system may be used over the course of a time period ranging from 1 second to 10 months, especially a period of 1 week to 5 months. The pregnancy monitoring system may be used between the first month of a pregnancy to the end of a pregnancy. For assessing likelihood of preterm labor and/or delivery, usage between the 20th month of a pregnancy and the 37th month of a pregnancy may be particularly beneficial.

In some cases, screening of patients may be performed to determine whether the pregnancy monitoring system is appropriate for their use. Said screening may include determining whether the patient is at a high risk of preterm birth (such as a patient that has had previous preterm births), has had previous miscarriages, and/or currently has her first pregnancy. Said screening may include determination of whether the patient is a good candidate for the pregnancy monitoring system. Said determination may include assessing the patient's weight, body mass index, the distance between the patient's belly surface and the uterus, and/or the number of fetuses that the mother is carrying. Screening may include assessing uterine electrical activity over a period of time, for example for assessing the amplitude of a uterine electrical activity signal.

Application of the data acquisition device may take place in a medical location such as doctor's office, in a non-medical location such as the patient's house, or in a combination thereof (for example, an initial data acquisition device may be applied in a doctor's office, and subsequent replacement data acquisition devices may be applied at the patient's home).

In some cases, the pregnancy monitoring system can be used by pregnant women at home or in the course of their everyday lives.

In some cases, the pregnancy monitoring system can be used by pregnant women within a medical care setting, for example within a hospital or clinic. In this use case, indications from said system may integrated with other monitoring system(s). Indications from said pregnancy monitoring system may be delivered to medical professionals via pager alert.

The pregnancy monitoring system may be used in combination with a fetal fibronectin test, cervical remodeling assessment, other anatomical, biochemical, and/or physiological assessments, and/or consideration of risk factors, to assess the likelihood of labor and/or delivery within a specified length of time or before a specified gestational age.

In some cases, the pregnancy monitoring system may be used in conjunction with a therapeutic system. Activation of said therapeutic system may comprise triggering based on likelihood of labor and/or delivery within a specified length of time or before a specified gestational age. Such triggering may be an automatic, semi-automatic, or manual process. Said therapeutic system may include a method and/or system for inducing labor, for example by delivery of pharmaceutical agents, transcutaneous electrical stimulation, and/or other means. Said therapeutic system may include a method and/or system for delaying labor and/or delivery. Delaying labor and/or delivery may be accomplished by delivery of pharmaceutical agents, transcutaneous electrical stimulation, and/or other means. For example, a tocolytic may be administered to delay delivery, then a corticosteroid may be administered to accelerate fetal development. In another example, a device may deliver electrical and/or mechanical inputs, such as stimulation or vibrations, that interrupt, counter, halt, arrest, cancel, or complement uterine contractions and/or associated electrical signaling or activity, in order to delay labor and/or birth.

In some embodiments, the electrodes of the pregnancy monitoring system require replacement at least once during the usage of the pregnancy monitoring system. In other embodiments, the electrodes of the pregnancy monitoring system do not require replacement over the course of usage of the pregnancy monitoring system.

In some cases, the data acquisition device is single use. In some cases, the data acquisition device is reusable.

In some embodiments, the data acquisition device may be water resistant. Some or all electronic components and/or wiring are fully isolated from direct contact with water. For example, analog signal processing circuitry, a microprocessor, a flexible printed circuit board, and wiring may be potted within silicone or epoxy. In some embodiments, said data acquisition device comprises an outer housing extending at least over the top, generally non-patient contacting side of said device, wherein the housing is made of a watertight biocompatible polymer.

In some embodiments, the patient contacting side of the data acquisition device has an adhesive member. In some cases, said adhesive member is comprised of a material that can maintain a bond to the patient's skin over a period of 1 day to 10 months with intermittent exposure to water, such during a shower. In some cases, said adhesive member comprises an acrylic adhesive. Said adhesive member may include a perimeter around the patient contacting side of the device, forming a water resistant barrier around the electrodes. Said adhesive member may comprise a perimeter around at least one individual electrode, forming a water resistant barrier.

In some cases, the pregnancy monitoring system may comprise a kit. The kit may contain more than one data acquisition device. In some cases, the kit may contain a sufficient number of data acquisition devices for monitoring the entire course of the patient's pregnancy. The kit may contain more than one data acquisition device with identical sensors. In some cases, the kit may contain data acquisition devices that are labeled to indicate when they are intended to be used. Said labeling may be by date, by week or month of pregnancy, by numerical labeling to indicate usage sequence, and/or by variation in color hue and/or intensity. The kit may contain data acquisition devices arranged in a sequence corresponding to the sequence in which they are intended to be used.

In some embodiments, at least one component of the pregnancy monitoring system contains a battery. In some cases, the battery can be accessed by the patient and replaced when necessary. In some cases, a charging port can be accessed by the patient and utilized to charge the battery when necessary. In some cases, the battery can be remotely charged by inductive charging. In some cases, said battery may not be directly accessed and/or charged, and after the battery is depleted the component is replaced and/or discarded.

Battery technologies used in the pregnancy monitoring system may include lithium-based battery chemistries (for example, lithium manganese dioxide, lithium silver vanadium oxide, lithium polymer, lithium carbon monofluoride, lithium iodine, and/or lithium iron phosphate).

In some embodiments, all electrodes of the pregnancy monitoring system are enclosed within a single housing of the data acquisition device. No external wires protrude from this housing, which may make said data acquisition device more comfortable for the patient to wear continuously for extended periods, including during sleep. In some embodiments, electrodes are enclosed within more than one housing; for example, a data acquisition device may be place near the patient's belly button, and a separate patch with a ground and/or reference electrode may be placed near the patient's hip.

In some embodiments, the data acquisition device protrudes minimally from the patient's belly. In some embodiments, protrusion in the ventral direction is minimized by variation in the thickness of the data acquisition device, for example with portions of the data acquisition device that are placed over the center of the belly having a smaller thickness than portions of the data acquisition device placed over more lateral regions of the belly. In some embodiments, utilization of flexible printed circuit boards contributes to conformability and minimizes protrusion.

In some cases, the pregnancy monitoring system comprises a data acquisition device with at least two electrodes. Such a data acquisition device may contain means for improved electrical coupling between the patient's skin and said electrodes, for example conductive gel and/or conductive elastomer. Migration of said gel may be constrained by at least one gel-constraining member. Such a data acquisition device may contain monopolar electrodes, bipolar electrodes, or a combination of monopolar and bipolar electrodes. Such a data acquisition device may contain mono and/or bi-dimensional arrays of electrodes. The inter-electrode distance of such a data acquisition device may be optimized to reduce the amplitude of cross-talk relative to target signals. Such a device may contain a flexible printed circuit board, may contain 2 to 1000 electrodes, and may contain at least one active electrode, with a low-noise preamplifier. Said at least one active electrode may be a ground and/or reference electrode.

In some cases, the pregnancy monitoring system contains a reference electrode. Said reference electrode may be placed at a location on the patient's surface that does not have as much uterine electrical activity as the patient's abdomen. Said reference electrode may be placed near the patient's hips.

The pregnancy monitoring system may contain means for double differential signal processing to reduce cross-talk between signals.

In some cases, the data acquisition device is configured to assess the contact impedance of at least one sensor. A notification may be triggered if said contact impedance is outside of a predetermined range. Said notification may be delivered to the patient, the patient's medical provider, a remote center, the company marketing the system, and/or a data monitoring platform. Said notification may be delivered in real time. In some cases, the pregnancy monitoring system is configured to assess the contact impedance of all sensors acquiring electrical uterine activity data. In some cases, the pregnancy monitoring system contains means for impedance matching for at least one sensor.

The pregnancy monitoring system may comprise a data acquisition device that attaches to a pregnant woman's abdomen, and communicates with a patient interface device and/or a remote center using radiofrequency communication. Measures may be implemented to reduce exposure of the fetus(es) and mother to potentially harmful radiation related to radiofrequency communication. For example: said radiofrequency communication may occur at lower frequencies, for example frequencies ranging from 100 kHz to 1 GHZ, and especially in the range of 10 MHz to 1 GHz; said radiofrequency communications may occur at low powers; said radiofrequency communications may be constrained to not exceed a predetermined threshold of time and/or energy per day; and/or data processing and/or analysis may be utilized to reduce the amount of data required to be transmitted to said patient interface device and/or said remote center.

In some cases, the pregnancy monitoring system contains at least two physically separate components that communicate via an encrypted communication channel. In some cases, the pregnancy monitoring system contains at least two physically separate components that enter an authentication procedure, for example before transmission of data, before entering a paired status, and/or at a periodic interval. In some cases, no data predetermined to be sensitive and/or access to system settings may be granted until authentication is successfully completed.

In some cases, the pregnancy monitoring system contains at least two components connected via a communication channel (for example, between a data acquisition device and a mobile telephone). Said components may not always be able to successfully communicate (for example, a patient may not always keep her mobile telephone near her data acquisition device). Therefore communications via said communications channel may follow a communications protocol designed to be interruption tolerant and/or minimize battery consumption. For example, one of said components may identify whether the other component is available for communication by periodically transmitting a preset signal and wait a predetermined time for a predetermined response sequence. If a said response is received, communications may begin; otherwise, said initiating component may enter a sleep mode. Each component may identify data that it intends to communicate and data that it has successfully communicated. Following an interruption in communications, for example due to excessive distance between a first device and a second device, the two devices may resume communications. After confirmation of successful communication, transmitted data may be deleted from the transmitting component to free memory space.

In some cases, one component of the pregnancy monitoring system may be lost or cease functioning properly. After this version has been replaced with a new version of the component, data may be transferred from other components of the pregnancy monitoring system to said new version via said communications channel. For example, a patient may lose a mobile phone used as part of the pregnancy monitoring system. After the patient replaces her phone with a new phone, her data acquisition device may transmit all previously acquired signals and analysis and/or a subset of previously acquired signals and analysis to said new phone.

In general, it should be understood that a data acquisition device and/or a patient interface device may be replaced during a pregnant woman's course of using the pregnancy monitoring system.

In some cases, a portable pregnancy monitor has an adhesive interface used to secure the portable pregnancy monitor to the skin of a pregnant female. In some cases, prior to use for monitoring, the adhesive interface is adhered to a backer. The backer may be comprised of paper, plastic, and/or other materials, and may be coated with a material that facilitates separation from the adhesive interface without significantly degrading the adhesive strength of the adhesive interface. The backer may be comprised of multiple sections, such that one section can be separated in full from the adhesive interface while another section remains, which may make handling of the portable pregnancy monitor easier. The backer may have tabs or regions that do not direction contact the adhesive interface, and allow the backer and/or regions of the backer to be easily peeled away from the adhesive interface. The portable pregnancy monitor with the backer may be contained or sealed in a dispenser that prevents damage, including degradation due to exposure to external air, air conditions (such as humidity or dryness) and/or temperature. In some cases, multiple dispensers may be attached to one another; in some cases, multiple portable pregnancy monitors and/or adhesive interfaces may be contained or sealed in a dispenser. In some cases, multiple adhesive interfaces may be affixed to one backer. The backer and dispenser configurations may also be used for adhesive patches that are stored separate from other components of the pregnancy monitor, but are used to secure the components to the pregnant female.

Components of the data acquisition device may be replaceable. In some cases, a patient may replace an adhesive that adheres said data acquisition device to her skin, while reusing some or all of the other components of the data acquisition device. In other cases, a patient may replace a patch-like component of the data acquisition device that comprises at least one electrode, an adhesive patient-contacting surface, and an electrical interconnection, while reusing a second component of the data acquisition device that comprises an electrical interconnection to connect with said patch-like component, a battery, means for signal processing, and means for delivering a notification to the patient or another person.

In some cases, the pregnancy monitoring system contains multiple components that can independently determine whether a notification should be delivered and deliver said notification (for example, regarding whether labor and/or delivery is likely to occur within a specified length of time or before a specified gestational age). This redundancy may assist in ensuring robustness of delivery of said notification, given potential communication channel failures and low battery issues.

In some cases, the pregnancy monitoring system contains at least one data acquisition device to be attached to the patient's abdomen. The maximum thickness of the device extending from the surface of the patient's skin, may range from 0.010″ to 3″, and especially range from 0.1″ to 1″.

In some cases, the pregnancy monitoring system contains a time-keeping means that adjusts to the correct local time. For example, said system may comprise a mobile telephone application that retrieves the correct local time from the mobile telephone. Said application may communicate the correct local time to other parts of said system, for example, to a data acquisition device. For example, said system may adjust notification time settings to reflect the time zone in which the notified parties are present.

In some cases, the data acquisition device may rely on movements of the wearer to partially or fully power the data acquisition device.

What is claimed is:

1. A method for monitoring the status of a pregnancy, the method comprising:
electrically coupling at least two electrodes to skin of a pregnant female using one or more adhesive patches;
receiving a processed uterine activity signal generated from measured uterine electrical activity;
computing from the processed uterine activity signal one or more of the following computed values: a conduction velocity associated with the uterine electrical activity; a peak frequency associated with the uterine electrical activity; a parameter associated with both the conduction velocity associated with the uterine electrical activity as well as the peak frequency associated with the uterine electrical activity;
comparing to a threshold value the one or more of the following: the conduction velocity associated with the uterine electrical activity; the peak frequency associated with the uterine electrical activity; the parameter associated with both the conduction velocity associated with the uterine electrical activity as well as the peak frequency associated with the uterine electrical activity; and
transitioning from a first mode to a second mode if the threshold value is exceeded by the one or more of the following: the conduction velocity associated with the uterine electrical activity; the peak frequency associated with the uterine electrical activity; the parameter associated with both the conduction velocity associated with the uterine electrical activity as well as the peak frequency associated with the uterine electrical activity;
wherein transitioning comprises enabling delivery of a notification related to a likelihood of onset of true labor, wherein delivery of the notification was not enabled in the first mode.

2. The method of claim 1, further comprising:
applying a first electric potential across an anatomical region spanned by a first set of electrodes comprising at least two electrodes, wherein the first set of electrodes is attached to the skin of the pregnant female using a first adhesive patch;
recording an impedance detected using the first set of electrodes;
replacing one or more of the at least two electrodes of the first set of electrodes with one or more replacement electrodes to create a second set of electrodes comprising at least two electrodes, wherein the one or more replacement electrodes of the second set of electrodes are electrically coupled to the skin of the pregnant female using a second adhesive patch;
applying a second electric potential across the anatomical region;
recording an impedance detected using the second set of electrodes; and
calculating a ratio between the impedance detected using the second set of electrodes and the impedance detected using the first set of electrodes.

3. The method of claim 2, further comprising using the ratio to achieve a consistent understanding or measurement, spanning the replacing of the one or more of the at least two electrodes of the first set of electrodes with one or more replacement electrodes to create the second set of electrodes, of one or more of the following: conduction velocity associated with uterine electrical activity; peak frequency associated with uterine electrical activity; a parameter associated with both conduction velocity associated with uterine electrical activity as well as peak frequency associated with uterine electrical activity.

4. The method of claim 3, further comprising:
identifying a first edge region of the first adhesive patch;
positioning a second edge region of the second adhesive patch in alignment with the first edge of the first adhesive patch; and
adhering the second adhesive patch to the skin of the pregnant female.

5. The method of claim 4, wherein an outline of the first edge region and second edge region after the alignment does not follow a straight line.

6. The method of claim 4, wherein the first edge region and the second edge region have features that geometrically interlock.

7. The method of claim 3, wherein the transitioning from the first mode to the second mode occurs after the replacing of the one or more of the at least two electrodes of the first set of electrodes with one or more replacement electrodes to create the second set of electrodes.

8. The method of claim 1, further comprising:
applying a first electric potential across an anatomical region spanned by a set of electrodes comprising at least two electrodes;
recording a first impedance detected using the set of electrodes comprising at least two electrodes;
replacing the first adhesive patch with a second adhesive patch;
applying, after replacing the first adhesive patch with the second adhesive patch, a second electric potential across the anatomical region;
recording a second impedance detected using the set of electrodes comprising at least two electrodes; and
calculating a ratio between the second impedance and the first impedance.

9. The method of claim 8, further comprising using the ratio to achieve a consistent understanding or measurement, spanning the replacing of the first adhesive patch with the second adhesive patch, of one or more of the following: conduction velocity associated with uterine electrical activity; peak frequency associated with uterine electrical activity; a parameter associated with both conduction velocity associated with uterine electrical activity as well as peak frequency associated with uterine electrical activity.

10. The method of claim 9, further comprising:
identifying a first edge region of the first adhesive patch;
positioning a second edge region of the second adhesive patch in alignment with the first edge of the first adhesive patch; and
adhering the second adhesive patch to the skin of the pregnant female.

11. The method of claim 10, wherein an outline of the first edge region and second edge region after the alignment does not follow a straight line.

12. The method of claim 9, wherein the transitioning from the first mode to the second mode occurs after the replacing of the first adhesive patch with a second adhesive patch.

13. The method of claim 1, wherein the likelihood of onset of true labor is the likelihood of onset of preterm labor.

14. The method of claim 1, wherein the likelihood of onset of true labor is the likelihood of onset of true labor within a specific interval.

15. The method of claim 1, wherein the one or more computed values is a parameter associated with a linear combination of the conduction velocity associated with the uterine electrical activity and the peak frequency associated with the uterine electrical activity.

16. The method of claim 1, wherein the one or more computed values is a parameter associated with a change of a linear combination of the conduction velocity associated with the uterine electrical activity and the peak frequency associated with the uterine electrical activity.

17. The method of claim 1, wherein the one or more computed values is a parameter associated with a rate of change of a linear combination of the conduction velocity associated with the uterine electrical activity and the peak frequency associated with the uterine electrical activity.

18. The method of claim 1, wherein the one or more computed values is a parameter associated with a change of the conduction velocity associated with the uterine electrical activity.

19. The method of claim 1, wherein the one or more computed values is a parameter associated with a change of the peak frequency associated with the uterine electrical activity.

20. The method of claim 3, wherein the one or more computed values is one or more of the following: a linear combination of the conduction velocity associated with the uterine electrical activity and the peak frequency associated with the uterine electrical activity; a change of a linear combination of the conduction velocity associated with the uterine electrical activity and the peak frequency associated with the uterine electrical activity; a rate of change of a linear combination of the conduction velocity associated with the uterine electrical activity and the peak frequency associated with the uterine electrical activity.
